(19) United States
(12) Patent Application Publication
(10) Pub. No.: US 2019/0352701 A1
(43) Pub. Date: Nov. 21, 2019

(54) METHOD FOR EVALUATING THE STATUS OF LATENT TUBERCULOSIS INFECTION, METHOD FOR EVALUATING THE EFFECT OF THE METHOD FOR TREATING THE SAME, AND TREATMENT METHOD THEREOF

(71) Applicant: Taichung Veterans General Hospital, Taichung (TW)

(72) Inventors: Tsai-Ling Liao, Taichung (TW); Der-Yuan Chen, Taichung (TW)

(21) Appl. No.: 16/144,571

(22) Filed: Sep. 27, 2018

(30) Foreign Application Priority Data
May 16, 2018 (TW) 107116691

(51) Int. Cl. C12Q 1/689; C12N 15/113; A61P 31/06; A61K 31/7105

(52) U.S. Cl. CPC C12Q 1/689; C12N 15/113; A61P 31/06; A61K 31/7105; C12Q 2600/106; C12Q 2600/158; C12Q 2600/118; C12Q 2600/112; C12Q 2600/178

(57) ABSTRACT

The present invention discloses a method for evaluating the status of latent tuberculosis infection, a method for evaluating the effect of a treatment therefor, and a treatment therefor. In the method for evaluating the status of latent tuberculosis infection, and the method for evaluating the effect of a treatment therefor, the expression level (s) of miR-889 and/or TWEAK are/is detected and used as a basis for analysis.

### BACKGROUND OF THE INVENTION

### 1. Field of the Invention

The present invention relates to latent tuberculosis infection-related technologies, and particularly to a method for evaluating the status of latent tuberculosis infection, a method for evaluating the effect of a method for treating the same, and a treatment method thereof.

### 2. Description of the Related Art

Tuberculosis is one of the most important infectious diseases in the world. Statistically, the annual incidence and mortality rate of tuberculosis in Taiwan are higher than those in other countries. According to the statistical data in 2015, there are 45.7 cases of tuberculosis and 2.4 cases of death per 100,000 people. The existing methods for diagnosis of active tuberculosis include sputum smear microscopy, bacterial culture, and imaging examination. Because Mycobacterium tuberculosis has a slow growth cycle, is not easy to culture, and is highly infectious, the detection of Mycobacterium tuberculosis needs to be carried out in a biosafety level 3 (BSL-3) laboratory, using a special medium for separation and purification, so that the existing methods for detecting active tuberculosis in clinic is unable to achieve real-time detection, and cannot be universally used in various medical institutions.

Furthermore, most patients with tuberculosis are in the status of latent tuberculosis infection (LTBI). When the patient is in the status of latent tuberculosis infection, the number of Mycobacterium tuberculosis in the body is too small to be diagnosed by chest imaging or bacterial culture. Currently, the tuberculin skin test and interferon-γ release assay are clinically used as the main diagnostic methods for latent tuberculosis infection. Specifically, the tuberculin skin test is convenient and less expensive. However, in countries where BCG vaccine is popular, false positives and lack of sensitivity may easily occur. The QFT-G (QuantiFERON-TB Gold) test used in interferon-γ release assay is not only expensive, but also cannot be used to distinguish latent tuberculosis infections from Active tuberculosis.

It can be seen from the above description that there is currently a lack of biomarkers and methods for rapidly detecting and diagnosing tuberculosis in real time.

### SUMMARY OF THE INVENTION

A first object of the present invention is to provide at least one novel biomarker, miR-889 and/or TWEAK, the expression of which is related to latent tuberculosis infection. That is, by detecting the expression or expression level of the biomarker in a sample, the risk of the sample provider having latent infection with M. tuberculosis, the prognosis of tuberculosis or the response to treatment can be evaluated or determined. Moreover, the expression or expression level of the biomarker detected in the sample can also be used as a criterion in screening drugs for treating tuberculosis.

A second object of the present invention is to provide pharmaceutical composition for treating or/and ameliorating latent tuberculosis infection, which comprises a miR-889 inhibitor as the active ingredient.

Therefore, in order to achieve the above object, an embodiment of the present invention discloses a method for detecting latent infection with Mycobacterium tuberculosis, which comprises the following steps:

detecting the expression level of at least one biomarker in a sample to be tested; and

analyzing the expression level of the biomarker in the sample to be tested to determine or evaluate the risk of a provider of the sample to be tested having latent infection with Mycobacterium tuberculosis.

In an embodiment of the present invention, the biomarker is miR-889. When the expression level of miR-889 in the sample to be tested is higher than the expression level of miR-889 in a normal sample, the provider of the sample to be tested is shown to have a high risk of latent infection with M. tuberculosis. When the expression level of miR-889 in the sample to be tested is lower than the expression level of miR-889 in a normal sample, the provider of the sample to be tested is shown to have a low risk of latent infection with M. tuberculosis.

In another embodiment of the present invention, the biomarker is TWEAK. When the expression level of TWEAK in the sample to be tested is higher than the expression level of TWEAK in a normal sample, the provider of the sample to be tested is shown to have a high risk of infection with M. tuberculosis or recurrence of latent tuberculosis infection. When the expression level of TWEAK in the sample to be tested is lower than the expression level of TWEAK in a normal sample, the provider of the sample to be tested is shown to have a low risk of infection with M. tuberculosis or recurrence of latent tuberculosis infection.

Another embodiment of the present invention discloses a method for evaluating whether a treatment is effective for latent tuberculosis infection, which comprises the following steps:

detecting the expression of a biomarker in a sample infected with Mycobacterium tuberculosis, to obtain a first expression level, where the biomarker is selected from the group consisting of miR-889 and TWEAK;

administering a treatment to the sample;

after the treatment is received, detecting the expression of the biomarker in the sample, to obtain a second expression level; and

comparing the first expression level with the second expression level, to determine whether the treatment is effective in treating latent tuberculosis infection, where when the biomarker is miR-889, if the second expression level is higher than or equal to the first expression level, the treatment is shown to have a poor therapeutic effect in treating latent tuberculosis infection, and if the second expression level is lower than the first expression level, the treatment is shown to have a good therapeutic effect in treating latent tuberculosis infection; and

when the biomarker is TWEAK, if the second expression level is higher than the first expression level, the treatment is shown to have a good therapeutic effect in treating latent tuberculosis infection, and if the second expression level is lower than or equal to the first expression level, the treatment is shown to have a poor therapeutic effect in treating latent tuberculosis infection in the sample provider.

When the sample is from a patient with latent tuberculosis infection, the method for evaluating whether a treatment is effective for latent tuberculosis infection can be used to evaluate the prognosis of the patient.

Specifically, when the expression level of miR-889 in the sample after receiving the treatment is higher than or equal to the expression level of miR-889 in the sample before receiving the treatment, the sample provider is shown to have a poor response to the treatment and have a poor prognosis; and when the expression level of miR-889 in the sample after receiving the treatment is lower than the expression level of miR-889 in the sample before receiving the treatment, the sample provider is shown to have a good response to the treatment and have a good prognosis.

Alternatively, when the expression level of TWEAK in the sample after receiving the treatment is lower than or equal to the expression level of TWEAK in the sample before receiving the treatment, the sample provider is shown to have a poor response to the treatment and have a poor prognosis; and when the expression level of TWEAK in the sample after receiving the treatment is higher than the expression level of TWEAK in the sample before receiving the treatment, the sample provider is shown to have a good response to the treatment and have a good prognosis.

Furthermore, in an embodiment of the present invention, a method for determining the progress of latent tuberculosis infection is disclosed, which comprises the following steps:

providing a sample to be tested and detecting the expression level of TWEAK or/and miR-889 in the sample;

providing the expression level of TWEAK or/and miR-889 in a normal sample; and

comparing the expression level of TWEAK or/and miR-889 in the sample to be tested with the expression level of TWEAK or/and miR-889 in the normal sample, to determine the disease progress of latent tuberculosis infection in a provider of the sample to be tested.

For example, when the expression level of miR-889 in the sample to be tested is higher than the expression level of miR-889 in the normal sample, the provider of the sample to be tested is indicated to be a patient with latent tuberculosis infection (LTBI); when the expression level of TWEAK in the sample to be tested is higher than the expression level of TWEAK in the normal sample, the provider of the sample to be tested is indicated to be a patient with recurring latent tuberculosis infection; when the expression level of miR-889 in the sample to be tested is lower than the expression level of miR-889 in the normal sample, the provider of the sample to be tested is indicated to have cured latent tuberculosis infection.

In embodiments of the present invention, the sample to be tested, the normal sample, and the sample are respectively blood.

In embodiments of the present invention, the method for detecting the biomarker in each of the samples is a method known to those of ordinary skill in the art to which the present invention pertains, such as ELISA, QRT-PCR, and the like.

Further, the overexpression of miR-889 is related to the pathogenesis of Mycobacterium tuberculosis. Therefore, in an embodiment of the present invention, a pharmaceutical composition is disclosed, which comprises an effective amount of a miR-889 inhibitor, and at least one pharmaceutically acceptable carrier. By administering the pharmaceutical composition of the present invention to an individual with latent tuberculosis infection, it is possible to alleviate or/and treat the condition of latent infection with Mycobacterium tuberculosis.

### BRIEF DESCRIPTION OF THE DRAWINGS

FIG. 1 shows the differential expressions of miRNAs, as assayed by next generation sequencing, in the peripheral blood mononuclear cells derived from subjects in a latent tuberculosis infection positive (LTBI(+)) group and in a latent tuberculosis infection negative (LTBI(−)) group, in which red indicates that the relative expression level is higher than the median of all samples, and green indicates that the relative expression level is lower than the median of all samples.

FIG. 2 shows the miR-889 expressions in peripheral blood mononuclear cells after infection with M. bovis BCG.

FIG. 3 shows the miR-889 expressions, as detected by QRT-PCR, in peripheral blood mononuclear cells derived from subjects in a LTBI(+) group and a LTBI(−) group.

FIG. 4 shows the miR-889 expressions, as detected by QRT-PCR, in the plasma of subjects from a LTBI (+) group, an NTM group, a LTBI (−) group, and a healthy control group

FIG. 5 shows that the miR-889 expression declines significantly 3 months after the subjects in the LTBI group receive treatment, and is parallel to clinical remission of the subjects in the LTBI group.

FIG. 6 shows the correlation between miR-889 and IFN-γ in peripheral blood mononuclear cells derived from subjects in the LTBI group.

FIG. 7A is a microphotograph showing a granuloma-like structure produced by peripheral blood mononuclear cells observed under an inverted microscope on Day 8 after infection with M. bovis BCG at a multiplicity of infection (MOI) of 0.1 (simulated latent tuberculosis infection model).

FIG. 7B shows the miR-889 expressions in peripheral blood mononuclear cells on Days 4, 6, and 8 of culturing after infection with M. bovis BCG.

FIG. 7C shows the bacterial growth in peripheral blood mononuclear cells on Days 4, 6, and 8 of culturing after infection with M. bovis BCG.

FIG. 8 shows the result of predicting the pairing of the genes TWEAK and miR-889.

FIG. 9A shows the luciferase activities in THP-1 cells transfected with various plasmids after treatment respectively with a miR-889 mimic, a control mimic, a miR-889 inhibitor, and a control inhibitor.

FIG. 9B shows the TWEAK mRNA expressions in THP-1 cells after various treatments.

FIG. 9C shows the TWEAK levels secreted from THP-1 cells after various treatments.

FIG. 10 shows the TWEAK levels secreted from peripheral blood mononuclear cells derived from RA patients with or without M. bovis BCG infection.

FIG. 11 shows the survival rate, as detected by NTT assay, of peripheral blood mononuclear cells derived from RA patients after various treatments.

FIG. 12A shows the TWEAK expressions in the serum of RA patients with latent tuberculosis infection, RA patients without latent tuberculosis infection, and healthy subjects.

FIG. 12B shows the TWEAK expressions in the serum of patients with latent tuberculosis infection before treatment.

FIG. 13A shows the miR-889 expressions in peripheral blood mononuclear cells from sample providers in various infection progress including latent tuberculosis infection (LTBI), recurrence of latent tuberculosis infection into active tuberculosis (TB reactivation), and completing the anti-tuberculosis treatment (Cured).

FIG. 13B shows the TWEAK mRNA expressions in peripheral blood mononuclear cells from sample providers in various infection status including latent tuberculosis infection (LTBI), recurrence of latent tuberculosis infection into active tuberculosis (TB reactivation), and completing the anti-tuberculosis treatment (Cured).

### DETAILED DESCRIPTION OF THE PREFERRED EMBODIMENTS

The present invention discloses a novel biomarker for detecting tuberculosis, which is miR-889 and/or TWEAK. Since the expression of the biomarker is associated with latent infection with Mycobacterium tuberculosis, the status of latent tuberculosis infection, and the prognosis of latent tuberculosis infection, the disease status of tuberculosis, the condition of tuberculosis infection, and the treatment outcome of tuberculosis in a sample provider can be evaluated or determined accurately in clinic by detecting the expression level of miR-889 or/and TWEAK in a sample.

In the following, in order to verify the efficacy of the present invention, the present invention will be further described with reference to several examples and accompanying drawings.

The clinical trials in the examples disclosed in the present invention were approved by the Ethics Committee of Taichung Veterans General Hospital (CE13330B), and the written consents of all subjects were got in accordance with the Declaration of Helsinki.

### Example 1: Clinical Test Results of Subjects

80 patients with rheumatoid arthritis (hereinafter referred to as RA patients) satisfying the 2010 revised criteria of the American College of Rheumatology (ACR) and 23 healthy subjects were enrolled. The disease activity of RA patients was evaluated according to the Disease Activity Score 28 joints (DAS28), and the DAS28 of RA patients enrolled in this example was greater than 3.2, of which 66 (82.5%) RA patients have received treatments with biologics in accordance with the guidelines of the British Society for Rheumatology, and the remaining 14 (17.5%) RA patients received only conventional synthetic disease-modifying anti-rheumatic drugs (csDMARDs). Moreover, the RA patients that were diagnosed as having active tuberculosis infection by bacterial culture or pathological examination were excluded.

All RA patients received standard interviews, physiologic examinations, and examination by chest imaging based on the standards for tuberculosis infection, and blood tests were performed by QFT-G analysis. When the results of QFT-G analysis show that IFN-γ is greater than or equal to 0.35 IU/ml, the test result is positive, indicating that the RA patient has latent tuberculosis infection and is a target of preventive treatment. The examination results of all RA patients are shown in Table 1, wherein the LTBI (+) group is a group having RA patients with latent tuberculosis infection, the NTM group is a group having RA patient with non-tuberculous mycobacterial (NTM) infection, and the LTBI(−) group is a group having RA patients without mycobacterial infection.

TABLE 1
Physiologic examination and interview results
of subjects in various groups

| | LTBI(+) group | NTM group | LTBI(−) group |
|---|---|---|---|
| | (n = 33) | (n = 12) | (n = 35) |
| Age of subject | 60.5 ± 13.4 | 62.1 ± 10.7 | 59.9 ± 12.2 |
| (years) | | | |
| Gender (female, %) | 24 (72.7) | 8 (66.7) | 27 (77.1) |
| Duration of disease | 9.4 ± 5.6 | 9.6 ± 4.5 | 10.6 ± 2.8 |
| (years) | | | |
| Complications | | | |
| Diabetes mellitus | 4 (12.1) | 2 (16.7) | 3 (8.6) |
| Chronic kidney | 4 (12.1) | 3 (25.0) | 5 (14.3) |
| disease | | | |
| Rheumatoid factor | 25 (75.8) | 5 (41.6) | 25 (71.4) |
| (positive, %) | | | |
| Cyclic | 30 (90.9) | 8 (66.7) | 25 (71.4) |
| citrullinated | | | |
| peptide (CCP) | | | |
| antibody | | | |
| (positive, %) | | | |
| DAS28 upon | 3.9 ± 1.5 | 3.8 ± 1.7 | 3.6 ± 0.2 |
| admission | | | |
| Erythrocyte | 28.7 ± 8.3 | 11.5 ± 3.5 | 16.4 ± 8.8 |
| sedimentation rate | | | |
| (mm/h) | | | |
| C-reactive protein | 0.95 ± 1.47 | 1.14 ± 1.15 | 0.98 ± 1.49 |
| (mg/dL) | | | |
| Prednisolone dose | 4.4 ± 2.8 | 4.7 ± 1.7 | 3.9 ± 1.8 |
| (mg/day) | | | |
| Methotrexate (%) | 20 (60.6) | 4 (33.3) | 15 (42.9) |
| Hydroxychloroquine | 17 (51.5) | 7 (58.3) | 8 (22.9) |
| (%) | | | |
| Sulfasalazine (%) | 7 (21.2) | 2 (16.7) | 4 (11.4) |
| Biologic therapy | 27 (81.8) | 9 (75.0) | 30 (85.7) |
| (%) | | | |
| Adalimumab | 12 (36.4) | 3 (25.0) | 12 (34.3) |
| Etanercept | 8 (24.2) | 4 (33.3) | 8 (22.9) |
| Rituximab | 6 (18.2) | 2 (16.7) | 7 (20.0) |
| Abatacept | 0 (0) | 0 (0) | 2 (5.7) |
| Tocilizumab | 1 (3.0) | 0 (0) | 1 (2.9) |

### Example 2: Isolation of miRNAs

Total RNA was extracted with the TRIzol reagent (Invitrogen, ThermoFisher 140 Scientific, USA), and purified by using the RNeasy MinElute Cleanup kit (QIAGEN, Germany). The purified RNA was quantified spectrophotometrically by determining the OD at 260 and 280 nm, and the isolated miRNAs were identified by capillary gel electrophoresis using Bioanalyzer 2100 (Agilent Technology, Palo Alto, Calif., USA). To verify the miRNAs extracted from plasma by QRT-PCR, a synthetic Caenorhabditis elegans miRNA (cel-miR-39, 147 Applied Biosystems, ThermoFisher Scientific, USA) was added as an internal control.

### Example 3: Quantitative Reverse Transcription Polymerase Chain Reaction (QRT-PCR)

The miRNA expressions in a sample were detected using the TaqMan miRNA detection zit (Applied Biosystems, ThermoFisher Scientific, USA) and QRT-PCR was performed in the StepOne Plus real-time PCR system. Small nuclear RNA (hereinafter referred to as RUN6) and synthetic cel-miR-39 (hereinafter referred to as cel-miR-39) were respectively used as an internal control gene in cells and plasma. The multiple expression of the target gene was obtained by calculating the average expression of the target gene relative to the internal control gene in the sample by comparative threshold cycle (Ct) method, and evaluated by 2dCt, where dCt is the average (Ct_{miRNAs gene}−Ct_{Rnu6/cel-miR-39}) of the healthy control group from which the average (Ct_{miRNAs gene}−Ct_{Rnu6/cel-miR-39}) of the disease group is subtracted.

### Example 4: Cell Culture

Peripheral blood mononuclear cells were isolated from venous blood by density gradient centrifugation using Ficoll-Paque PLUS (GE Healthcare Biosciences AB, Uppsala, Sweden). The peripheral blood mononuclear cells and human monocyte cell line (hereinafter referred to as THP-1 cells, ATCC TIB-202) were respectively cultured in RPMI medium 1640 (Gibco, ThermoFisher Scientific, USA) supplemented with 10% fetal bovine serum, lx non-essential amino acids, 100 unit/ml penicillin and 100 unit/mi streptomycin in an environment having 5% carbon dioxide at 37° C. To induce the cells to differentiate into macrophages, the THP-1 cells (1×10⁶ cells/mL) were cultured in RPMI medium and treated with 10 ng/mi PMA (phorbol myristate acetate, Sigma, USA) overnight.

### Example 5: Differential Expressions of miRNAs Assayed by Next Generation Sequencing

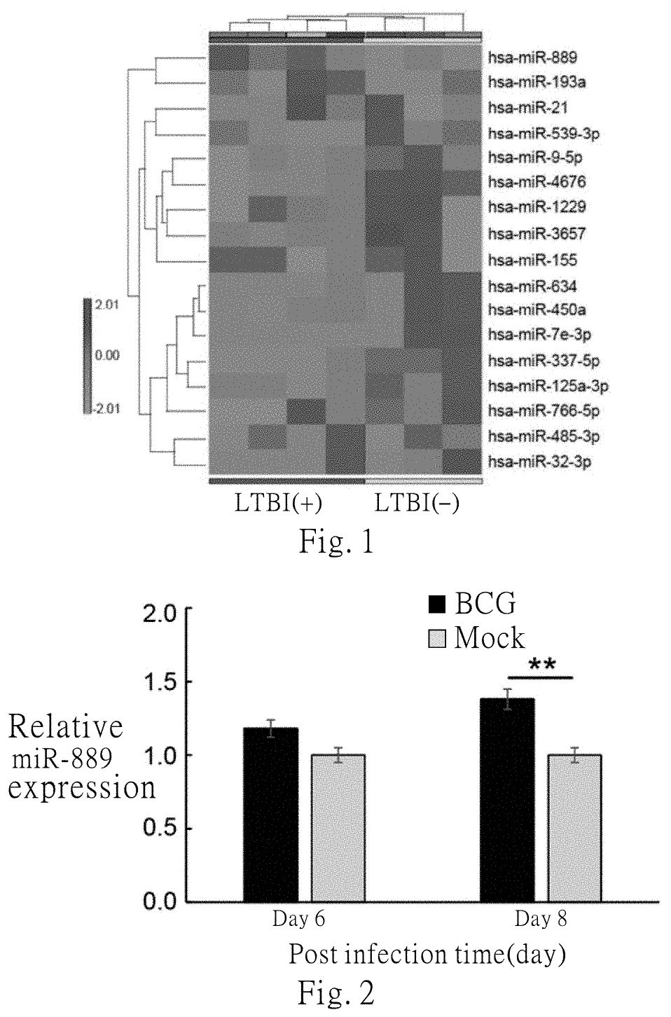

The peripheral blood mononuclear cells were respectively derived from the subjects in the LTBI(+) group and the LTBI(−) group, and assayed for miRNA expressions by next generation sequencing. The results are shown in FIG. 1 and Table 2. When the value of fold changes in miRNA expressions is greater than 3.00 or less than 0.330, the difference is considered to be significant.

The miRNA expressions were assayed by next generation sequencing (NGS) as follows. A small RNA library was constructed using the total RNA-Seq kit v2.0 (ThermoFisher Scientific, USA) and a template was prepared with the Ion PGM Template OT2 200 kit (ThermoFisher Scientific, USA). The Ion PGM Template OT2 200 kit and a chip were used with the Ion PGM sequencer. Alignment with the human hg19 genome was performed by pairing software (built-in Torrent Suite software v4.0, ThermoFisher Scientific, USA) and the differential expressions was assayed with Partek Genomic Suite 6.6 (Partek).

TABLE 2
miRNA expressions, as assayed by next
generation sequencing, in peripheral blood mononuclear
cells derived from subjects in LTBI(+) group and LTBI(−)
group

| Up-regulated | Fold change | Dow-regulated | Fold change |
|---|---|---|---|
| miRNAs | (median) | miRNA | (median) |
| has-miR-889 | 4.02 | has-miR-9-5p | 0.32 |
| has-miR-193a | 3.03 | has-miR-4676 | 0.30 |
| has-miR-21 | 2.22 | has-miR-1229 | 0.20 |
| has-miR-539-3p | 2.40 | has-miR-3657 | 0.32 |
| has-miR-941 | 2.14 | has-miR-634 | 0.28 |
| has-miR-485-3p | 2.32 | has-miR-450a | 0.28 |
| | | has-miR-7e-3p | 0.31 |
| | | has-miR-337 | 0.20 |
| | | has-miR-125a-3p | 0.27 |
| | | has-miR-766-5p | 0.24 |
| | | has-miR-32-3p | 0.16 |

The results in FIG. 1 and Table 2 show that a total of 17 miRNAs have significant differential expressions in peripheral blood mononuclear cells, of which 6 miRNAs in the LTBI (+) group are positively regulated and 11 miRNAs are negatively regulated, compared to the LTBI(−) group.

### Example 6: Mycobacterium tuberculosis Induces the Peripheral Blood Mononuclear Cells to Express miR-889

M. bovis BCG was cultured in Middlebrook 7H11 medium in an environment containing 5% carbon dioxide 37° C.

The peripheral blood mononuclear cells were divided into two groups, one of which was infected with M. bovis BCG (MOI=0.1), and the other group was a blank group. The expression levels of miR-889 in the cells were detected respectively after 6 and 8 days of culture. The results are shown in FIG. 2.

The results in FIG. 2 show that the expression level of miR-889 is increased in the peripheral blood mononuclear cells infected with M. bovis BCG, and the the expression level of miR-889 is increased with the elapse of time after infection.

### Example 7: Verification by QRT-PCR of miR-889 being a Biomarker for Tuberculosis

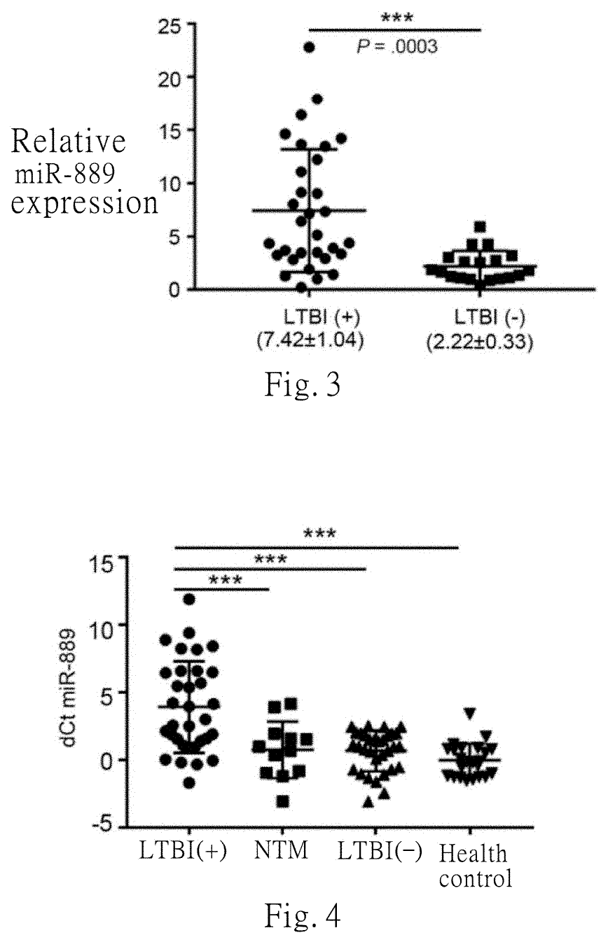

The miR-889 expressions in peripheral blood mononuclear cells derived from subjects in the LTBI(+) group and the LTBI (−) group were detected by QRT-PCR. The results are shown in FIG. 3. The miR-889 expressions in the plasma of subjects from the LTBI(+) group, the NTM group, the LTBI(−) group, and the healthy control group were detected by QRT-PCR, in which the healthy control group was used as a control group. The results are shown in FIG. 4.

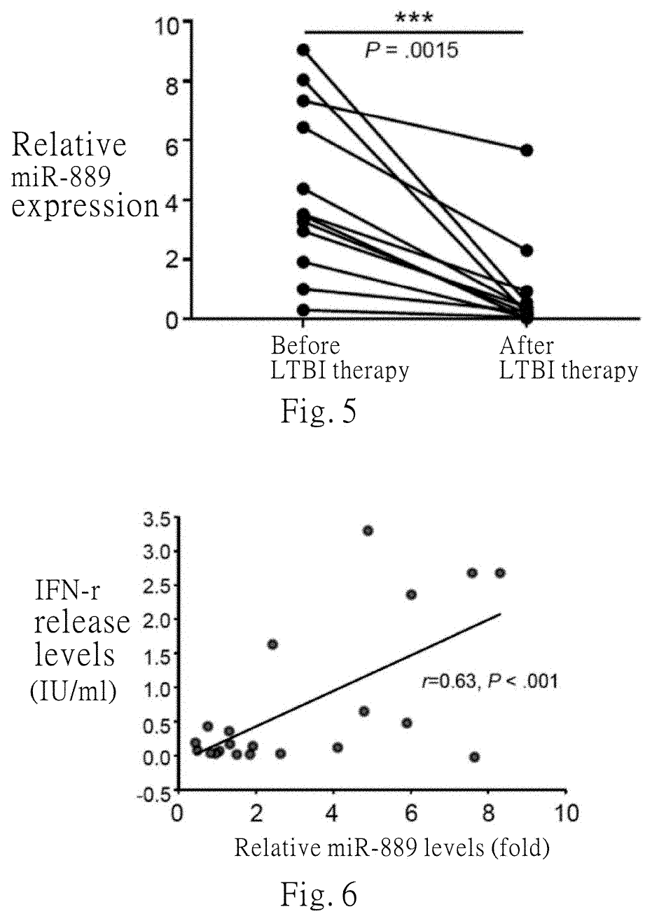

In addition, after receiving treatment with the drugs rifapentine and isoniazid for 3 months, the subjects in the LTBI groups were tested for miR-889 expression. The results are shown in FIG. 5. The correlation between IFN-γ and miR-889 was detected by QFT-G analysis. The results are shown in FIG. 6.

As can be known from the results in FIG. 3, when the sample provider is a latent tuberculosis infection (LTBI) the expression level of miR-889 in peripheral blood mononuclear cells derived therefrom is 7.42±1.04 folds, which is higher than that in the non-infected sample provider (2.22±0.33 time). As can be known from the results in FIG. 4, the expression level of miR-889 (dCt 3.94±0.59) in the plasma of the subjects in the LTBI (+) group is obviously higher than the expression level of miR-889 (dCt 0.77±0.60) in the plasma of the subjects in the NTI4 group, the expression level of miR-889 (dCt 0.68±0.27) in the plasma of the subjects in the LTBI (−) group, and the expression level of miR-889 (dCt 0.00±0.26) in the plasma of the subjects in the healthy control group.

As can be known from the results in FIGS. 5 and 6, after receiving the treatment with drugs, the expression level of miR-889 in peripheral blood mononuclear cells derived from the subjects in the LTBI (+) group is decreased from 4.31±0.81 folds by 0.92±0.47 folds, and the expression level of miR-889 is positively correlated with the release of IFN-γ.

It can be seen that miR-889 can be used as a biomarker in evaluating the prognosis of and therapeutic effect in patients having latent infection with Mycobacterium tuberculosis. In other words, after a same provider receives an anti-latent tuberculosis infection therapy, if the expression level of miR-889 cannot be reduced after treatment, it indicates that the sample provider has a poor prognosis or the therapy is ineffective.

### Example 8: Detection of miR-889 Expression in Human Tuberculosis Granuloma Model

M. bovis BCG was cultured as described in Example 6.

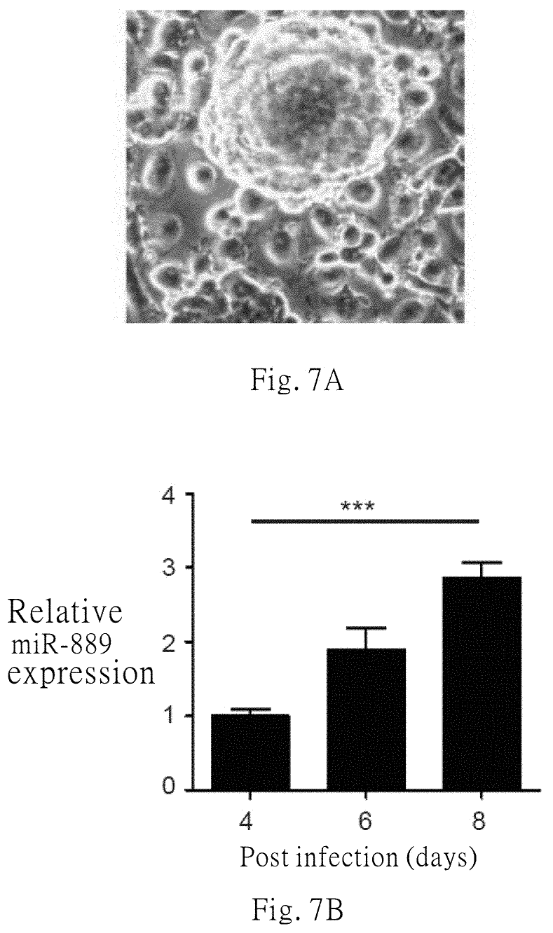

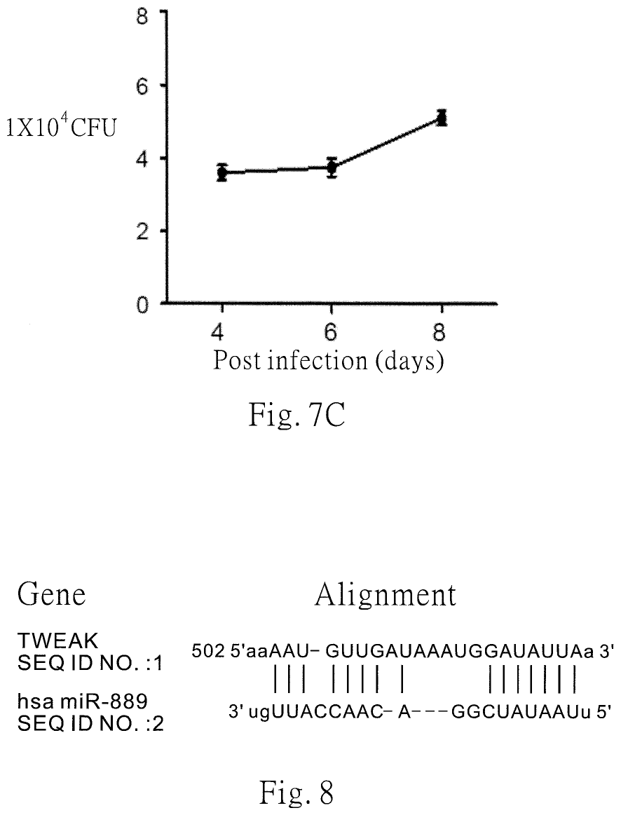

Freshly isolated peripheral blood mononuclear cells were infected with M. bovis BCG at MOI of 0.1 (simulated latent tuberculosis infection model), followed by culturing for 11 days in RPMI medium 1640 supplemented with 20% human serum in an environment containing 5% carbon dioxide at 37° C., during which the serum and medium were changed every 4 days. The granuloma-like structure was lysed at Days 4, 6 and 8 after infection, and the lysate was diluted and cultured on Middlebrook 7H11 medium. The miR-889 expression and the number of colonies were detected. The results are shown in FIG. 7.

As can be known from the results shown in FIG. 7, the miR-889 expression is 1.00±0.08 folds on Day 4 after infection with M. bovis ECG, and the miR-889 expression is increased to 2.85±0.21 folds on Day 8 after infection with M. bovis BCG. This result shows that the expression level of miR-889 increases with the formation of the granuloma-like structure and the growth of M. tuberculosis, indicating that miR-889 can be used as a biomarker for latent tuberculosis infection and determining the disease status of tuberculosis.

### Example 9: TWEAK is a Target of miR-889

A sequence at the 3′UTR end of human TWEAK (tumor necrosis factor-like weak inducer of apoptosis) was cloned into a pMIR-REPORT luciferase vector, to construct a TWEAK WT luciferase reporter plasmid.

Bio-information analysis predicts that the TWEAK (tumor necrosis factor-like weak inducer of apoptosis) (gene bank: NM 003809)(SEQ ID No.: 1) is a target of miR-889 SEQ ID No.:2), and the 3′ UTR end thereof can be paired with miR-889, as shown in FIG. 8. The predicted binding sites in the 3′UTR end of TWEAK was further mutated to construct a TWEAK MT luciferase reporter plasmid, which was used as a control group.

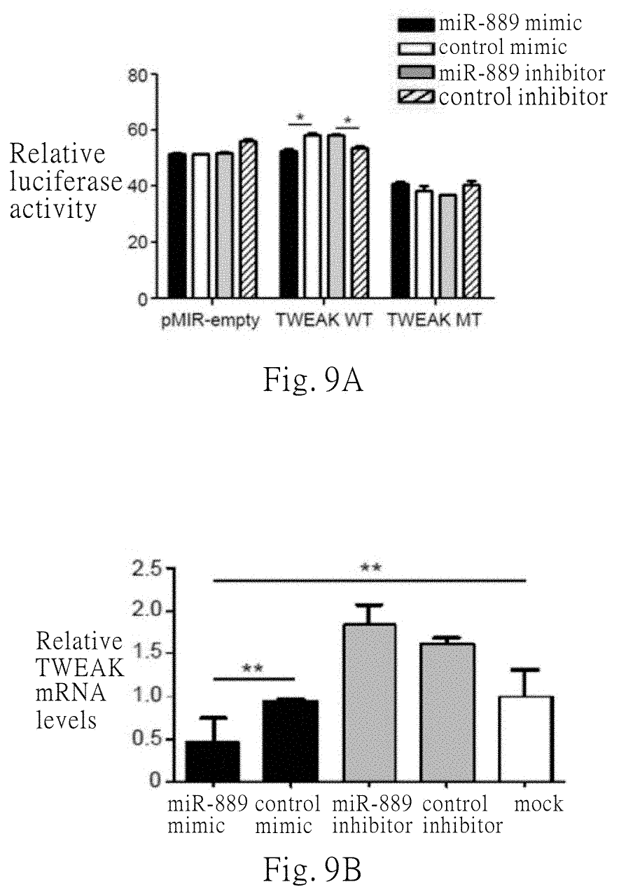

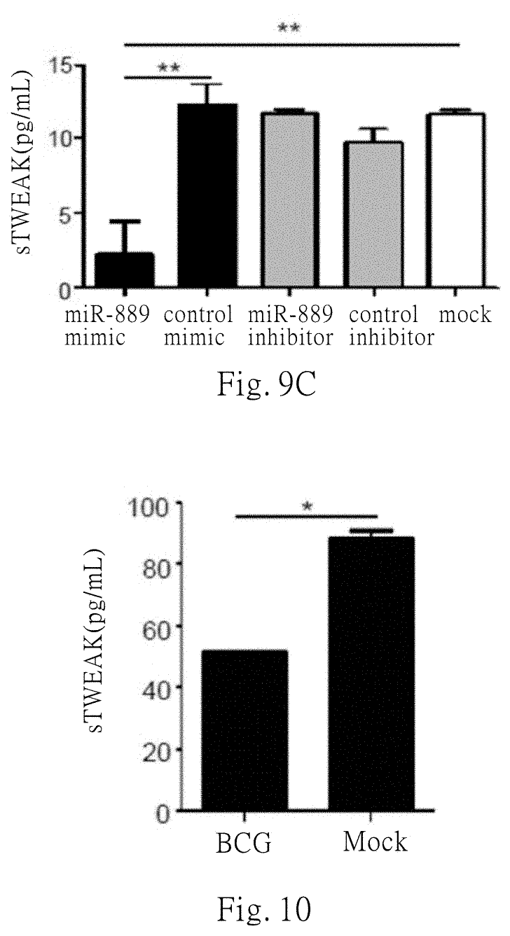

The pMIR-REPORT luciferase vector (with no TWEAK cloned), the TWEAK WT luciferase reporter plasmid, and the TWEAK MT luciferase reporter plasmid were transfected into THP-1 cells, respectively, and then treated and cultured with a miR-889 mimic, a control mimic, a miR-889 inhibitor, and a control inhibitor separately. The luciferase activity, TWEAK mRNA expression and TWEAK secretion were measured. The results are shown in FIGS. 9A to 9C.

As can be seen from the results in FIG. 9A, compared with the control inhibitor, the miR-889 inhibitor increases the luciferase activity in the cells transfected with the TWEAK WT luciferase reporter plasmid, and the miR-889 mimic reduces the luciferase activity in the cells transfected with the TWEAK WT luciferase reporter plasmid.

As can be seen from the results in FIGS. 9B and 9C, in the THP-1 cells treated with the miR-889 mimic to overexpress miR-889, the TWEAK mRNA expression is 0.49±0.26 fold, and the amount of TWEAK secreted is 2 2±2 6 pg/ml; in the THP-1 cells treated with the control mimic, the TWEAK mRNA expression is 0.95±0.03 folds, and the amount of TWEAK secreted is 12.2±1.5 pg/ml; and in the non-transfected THP-1 cells, the TWEAK mRNA expression is 1.01±0.02 folds, and the amount of TWEAK secreted is 11.6±0.3 pg/ml. It can be known that compared with the cells receiving no treatment or treated with the control mimic, the TWEAK expression level in the cells overexpressing miR-889 is significantly reduced.

Therefore, according to the results shown in FIG. 9, TWEAK is indeed a target of miR-889, and the overexpression of miR-889 will reduce the TWEAK expression, that is, TWEAK is negatively regulated by miR-889.

### Example 10: Detection of TWEAK Expression Level in Human Tuberculosis Granuloma Model

As described in Example 10, peripheral blood mononuclear cells were isolated from RA patients, and then infected with M. bovis BCG at an MOI of 0.1 (simulated latent tuberculosis infection model), respectively. On day 8 after infection (the granuloma structure was formed), the TWEAK expression was examined. The results are shown in FIG. 10.

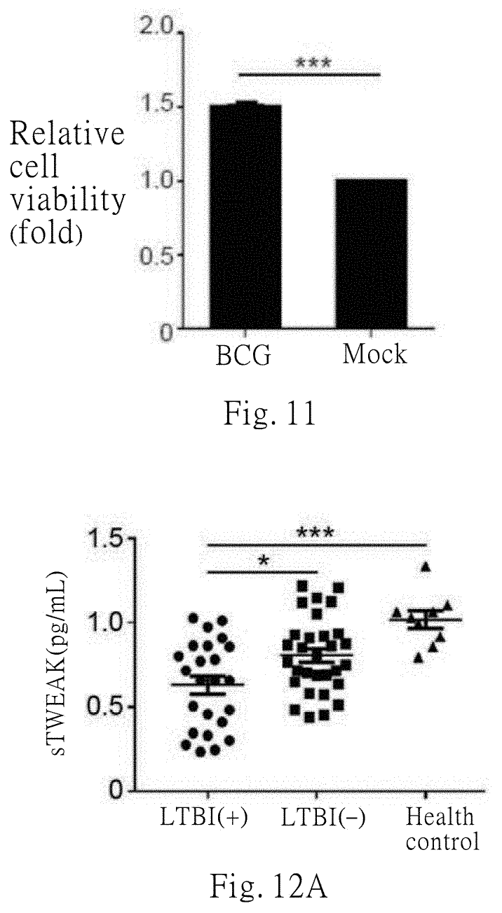

The survival rates of peripheral blood mononuclear cells infected with or without M. bovis BOG was detected by NTT assay. The results are shown in FIG. 11.

As can be known from the results in FIG. 10, the amount of TWEAK secreted by cells infected without M. bovis is 88.7±1.79 pg/ml, and the amount of TWEAK secreted by cells infected with M. bovis is significantly reduced (51.7±0.13 pg/M)), showing that the latent infection with M. bovis will cause a decrease in the amount of TWEAK secreted by the cells. As can be known from the results in FIG. 11, the survival rate of cells infected with M. bovis is 1.5 times that of cells infected without M. bovis. As can be known from the results in FIGS. 10 and 11, there is a negative correlation between the TWEAK expression level and the cell survival rate.

### Example 11: Relationship Between TWEAK Expression and Tuberculosis Infection

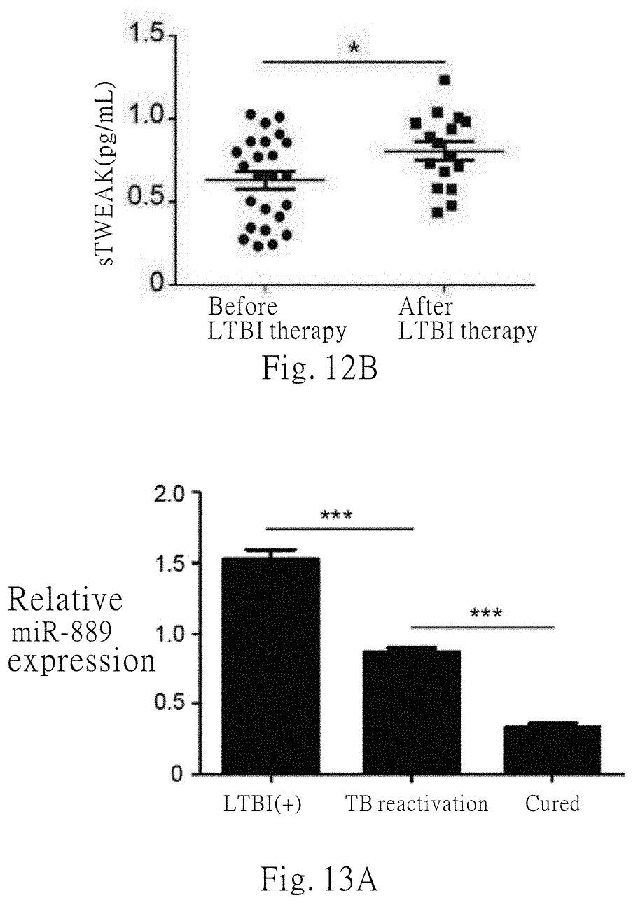

The TWEAK expressions in the serum of RA patients with latent tuberculosis infection (LTBI(+)), RA patients without infection (LTBI(−)), and healthy subjects were detected. The results are shown in FIG. 12A.

As can be known from the results shown in FIG. 12A, the expression level of TWEAK in RA patients with latent tuberculosis infection is 0.63±0.05 ng/ml; the expression level of TWEAK in RA patients without infection is 0.81±0.04 ng/ml; and the expression of TWEAK in healthy subjects is 1.02±0.05 ng/ml.

The TWEAK expressions in the serum of RA patients with latent tuberculosis infection before and after receiving an anti-latent tuberculosis therapy were detected. The results are shown in FIG. 12B.

As can be known from the results shown in FIG. 12B, the expression level of TWEAK in RA patients with latent tuberculosis infection is 0.63±0.05 ng/ml before treatment, and is 0.8110.06 ng/ml after treatment.

In combination with the results of Examples 9 to 11, it can be seen that even if the sample provider is in a status of latent infection with Mycobacterium tuberculosis, the expression level of TWEAK in blood still changes. That is, the risk of a sample provider having latent infection with M. tuberculosis can be determined or evaluated by detecting the TWEAK expression in a sample. When the expression level of TWEAK in the sample is below a predetermined normal value, the sample provider is indicated to have a high risk of latent tuberculosis infection. Conversely, when the expression level of TWEAK in the sample is equal or close to a predetermined normal value, the sample provider is indicated to have a low risk of latent tuberculosis infection. Furthermore, since the expression level of TWEAK can be used for detecting the condition of infection with Mycobacterium tuberculosis in a sample provider, the response of the sample provider to a predetermined treatment and the prognosis can be evaluated by detecting and comparing the expression levels of TWEAK in samples before and after the sample provider receives the predetermined treatment.

### Example 12: miR-889 and TWEAK are Useful as Biomarkers for Determining the Status and Prognosis of Latent Tuberculosis Infection

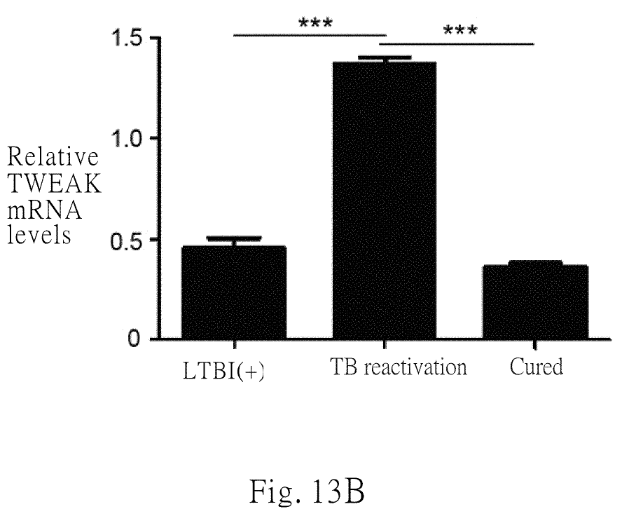

One of the enrolled subjects in the LTBI groups developed pulmonary tuberculosis complicated by ascites after receiving treatment with Adalimumab for 4 months. The patient was treated with a anti-tuberculosis therapy, and confirmed to complete the treatment by chest imaging and bacterial culture. The miR-889 and TWEAK mRNA expressions in peripheral blood mononuclear cells derived from the patient in the status including latent tuberculosis infection, recurrence of latent tuberculosis infection into active tuberculosis, and completing the anti-tuberculosis treatment were detected. The results are shown in FIGS. 13A and 13B.

As can be known from the results in FIG. 13A, the expression level of miR-889 is the highest in the peripheral blood mononuclear cells when the patient is in the status of latent tuberculosis infection, and is 1.52±0.07 folds; the expression level of miR-889 in peripheral blood mononuclear cells is the medium when the patient is in the status of recurrence of latent tuberculosis infection into active tuberculosis, and is 0.87±0.03 fold; and when the sample provider is treated with an anti-tuberculosis therapy and clinically tested to be tuberculosis negative, the expression level of miR-889 in peripheral blood mononuclear cells is the lowest (0.33±0.03 fold).

It can be seen that the miR-889 expression in a sample is detected, when the expression level of miR-889 in the sample is higher than a normal expression level, the sample provider is indicated to have a high risk of latent tuberculosis infection (LTBI); when the the expression level of miR-889 in the sample is lower than the normal expression level, the sample provider is indicated to have a low risk of latent tuberculosis infection (LTBI). Therefore, miR-889 can be used as a biomarker for detecting latent tuberculosis infection.

In addition, as can be known from the results shown in FIG. 13B, the biomarker is TWEAK, and when the sample provider is in the status of recurrence of latent tuberculosis into active tuberculosis, the expression level of TWEAK mRNA in peripheral blood mononuclear cells is the highest and is 1.37±0.03 folds; when the sample provider is in the status of latent tuberculosis infection, the expression level of TWEAK mRNA in peripheral blood mononuclear cells is the medium and is 0.46±0.05 fold; and when the sample provider is treated with an anti-tuberculosis therapy and clinically tested to be tuberculosis negative, the expression level of TWEAK mRNA in peripheral blood mononuclear cells is the lowest (0.36±0.02 fold).

It can be seen that the TWEAK expression in a sample is detected, when the expression level of TWEAK in the sample is higher than the expression level of TWEAK in a normal sample, the sample provider is shown to have a high risk of recurrence of latent tuberculosis infection into active tuberculosis; and when the expression level of TWEAK in the sample is lower than the expression level of TWEAK in the normal sample, the sample provider is shown to have a low risk of recurrence of latent tuberculosis infection. Therefore, TWEAK can be used as a biomarker for detecting the recurrence of latent tuberculosis infection.

What is claimed is:

1. A method for treating latent infection with M. tuberculosis, comprising administering an effective amount of a miR-889 inhibitor or a TWEAK promoter to a subject having latent tuberculosis infection, to treat the condition of infection with M. tuberculosis in the subject.

2. A method for evaluating whether a treatment is effective for latent tuberculosis infection, comprising the following steps:
detecting the expression of a biomarker in a sample infected with Mycobacterium tuberculosis, to obtain a first expression level, where the biomarker is selected from the group consisting of miR-889 and TWEAK;
administering a treatment to the sample;
after the treatment is received, detecting the expression of the biomarker in the sample, to obtain a second expression level; and
comparing the first expression level with the second expression level, to determine whether the treatment is effective in treating latent tuberculosis infection, wherein when the biomarker is miR-889, if the second expression level is higher than or equal to the first expression level, the treatment is shown to have a poor therapeutic effect in treating latent tuberculosis infection, and if the second expression level is lower than the first expression level, the treatment is shown to have a good therapeutic effect in treating latent tuberculosis infection; and
when the biomarker is TWEAK, if the second expression level is higher than the first expression level, the treatment is shown to have a good therapeutic effect in treating latent tuberculosis infection, and if the second expression level is lower than or equal to the first expression level, the treatment is shown to have a poor therapeutic effect in treating latent tuberculosis infection in the sample provider.

3. The method for evaluating the prognosis of patients having latent tuberculosis infection according to claim wherein the sample is blood.

4. The method for evaluating whether a treatment is effective for latent tuberculosis infection according to claim 2, wherein the sample is derived from a patient having latent tuberculosis infection.

5. The method for evaluating whether a treatment is effective for latent tuberculosis infection according to claim 2, wherein the treatment is medical treatment.

6. A method for determining the status and risk of latent tuberculosis infection, comprising the following steps:
detecting the expression level of a biomarker in a sample to be tested, wherein the biomarker is selected from the group consisting of miR-889 and TWEAK;
detecting the expression level of the biomarker in a normal sample; and
comparing the expression level of the biomarker in the sample to be tested with the expression level of the biomarker in the normal sample, to determine the status and risk of latent tuberculosis infection in a provider of the sample to be tested.

7. The method for determining the status and risk of latent tuberculosis infection according to claim 6, wherein when the expression level of miR-889 in the sample to be tested is higher than the expression level of miR-889 in the normal sample, the provider of the sample to be tested is shown to have a high risk of latent infection with M. tuberculosis; and when the expression level of miR-889 in the sample to be tested is lower than the expression level of miR-889 in the normal sample, the provider of the sample to be tested is shown to have a low risk of latent infection with M. tuberculosis.

8. The method for determining the status and risk of latent tuberculosis infection according to claim 6, wherein when the expression level of TWEAK in the sample to be tested is higher than the expression level of TWEAK in the normal sample, the provider of the sample to be tested is shown to have a high risk of infection with M. tuberculosis or recurrence of latent tuberculosis infection; and when the expression level of TWEAK in the sample to be tested is lower than or equal to the expression level of TWEAK in the normal sample, the provider of the sample to be tested is shown to have a low risk of infection with M. tuberculosis or recurrence of latent tuberculosis infection.

9. The method for determining the status and risk of latent tuberculosis infection according to claim 6, wherein when the expression level of miR-889 in the sample to be tested is higher than the expression level of miR-889 in the normal sample, the provider of the sample to be tested is indicated to be a patient with latent tuberculosis infection (LTBI); when the expression level of TWEAK in the sample to be tested is higher than the expression level of TWEAK in the normal sample, the provider of the sample to be tested is indicated to be a patient with recurring latent tuberculosis infection; and when the expression level of miR-889 in the sample to be tested is lower than the expression level of miR-889 in the normal sample, the provider of the sample to be tested is indicated to have cured latent tuberculosis infection.

10. The method for determining the status of latent tuberculosis infection according to claim 6, wherein the sample to be tested is blood.


SEQUENCE LISTING (2 sequences)

SEQ ID NO: 1 - DNA, 25 bp, human
  misc_feature (6)..(6): n is a, c, g, t or u
aaaaunguugauaaauggauauuaa

SEQ ID NO: 2 - RNA, 25 nt, human
  misc_feature (11)..(13): n is a, c, g, or u
  misc_feature (15)..(15): n is a, c, g, or u
uuaauaucggnnnancaaccauugu